(19) United States
(12) Patent Application Publication
(10) Pub. No.: US 2024/0024413 A1
(43) Pub. Date: Jan. 25, 2024

(54) COMPOSITION AND METHOD FOR TREATMENT OF ISCHEMIC DISEASE

(71) Applicants: Chun-Che SHIH, Taipei (TW); Chunhan SHIH, Pleasant Hill, CA (US)

(72) Inventors: Chun-Che SHIH, Taipei (TW); Tsai-Mu CHENG, Taipei City (TW); Hsin-Ying LU, Tainan City (TW); Chunhan SHIH, Pleasant Hill, CA (US); Fwu-Long MI, Taipei City (TW); Kuo-Tang TSENG, Taipei (TW)

(73) Assignees: Chun-Che SHIH, Taipei (TW); Chunhan SHIH, Pleasant Hill, CA (US)

(21) Appl. No.: 18/038,686

(22) PCT Filed: Dec. 2, 2020
(86) PCT No.: PCT/US2020/062809

§ 371 Date: May 24, 2023

(51) Int. Cl. A61K 38/16; A61K 47/69; B82Y 5/00; A61P 9/10

(52) U.S. Cl. CPC A61K 38/16; A61K 47/6935; A61K 47/6939; B82Y 5/00; A61P 9/10

(57) ABSTRACT

The present application provides a composition and a method for treating ischemia disease. The composition for treatment of ischemia disease comprises an angiopeptide comprising an amino acid sequence shown as any of SEQ ID Nos: 1 to 3; and a carrier for carrying the angiopeptide. The carrier can be a nanoparticle, a hygrogel or a combination thereof. The method for treatment of ischemia disease comprises administering the composition to a subject.

### BACKGROUND OF THE INVENTION

### 1. Field of the Invention

The present invention relates to a pharmaceutical composition and a method, and more particularly to a pharmaceutical composition and a method for treatment of ischemia.

### 2. Description of the Related Art

Ischemic heart disease (IHD), specifically acute myocardial infarction (AMI), is the leading cause of morbidity and mortality in the world. Ischemia and hypoxia are the main pathological process in MI, subsequently causing myocardial apoptosis, inflammation, arrhythmia, myocardial fibrosis, cardiac remodeling, and heart failure. Therefore, effective revascularization is of utmost importance. Therapeutic angiogenesis is based on the premise that the development of new blood vessels can be augmented by exogenous administration of the appropriate growth factors. Over the last years, results of preclinical studies on therapeutic angiogenesis for myocardial ischemia have provided inconsistent results and the definite means of inducing clinically useful therapeutic angiogenesis remain elusive. More studies are required to gain further insights into the biology of angiogenesis and address pharmacological limitations of current approaches of angiogenic therapy. Additionally, means of non-invasive individualized pharmacological therapeutic neovascularization may be the next major advance in the treatment of ischaemic heart disease.

Therapeutic angiogenesis involves exogenously administering an agent that stimulates the postnatal growth of new blood vessels to restore circulation to the tissue. Recent angiostudies have revealed postnatal vasculogenesis, as endothelial progenitor cells (EPCs) recruited from the bone marrow may circulate in the peripheral blood and become incorporated into sites of injury and can either differentiate into mature ECs or regulate pre-existing ECs via paracrine/juxtacrine signaling, which makes EPCs a favorable candidate for therapeutic studies. However, the promising potential of such growth factors has not yielded much clinical success. The principal limitation of proteins is the short half-life of exogenous proteins in target tissue, which reduces the therapeutic benefit. Therefore, the problem of sustained expression of the growth factors should be solved.

According to the above, novel therapeutic strategies are still highly desirable.

### SUMMARY

The present application provides a method for treating ischemia disease in a subject comprising administering a composition to the subject, wherein the composition comprises an angiopeptide comprising an amino acid sequence shown as any of SEQ ID Nos: 1 to 3.

The present application also provides a composition for treatment of ischemia disease comprising an angiopeptide comprising an amino acid sequence shown as any of SEQ ID Nos: 1 to 3; and a carrier for carrying the angiopeptide. The carrier can be a nanoparticle, a hygrogel or a combination thereof.

### BRIEF DESCRIPTION OF THE DRAWINGS

FIG. 1 shows the simulation structure of the angiopeptide in present application and the structure of Il-2R active site.

FIG. 2 shows the development of ischemia treatment of the present application.

FIG. 3 shows angiogenesis ability of the angiopeptide.

FIG. 4 shows successful preparation of the MI model.

FIG. 5 shows ameliorated myocardial infarct size by the angiopeptide, (A) the representative histological and immunohistochemical staining, and (B) the expression of p-ERK/ERK, p-IkB/IkB, and p-NF-kB/NF-kB in H9C2 cells treated with the angiopeptide.

FIG. 6 shows inhibition of oxidative stress by the angiopeptide, (A) SOD activity, (B) MDA level in the heart homogenates from LAD ligated rats, and (C) H₂O₂-induced mitochondrial ROS production in H9C2 cells.

FIG. 7 shows (A) MFP lowered monocyte (CD68) infiltration and increased infiltration of angiogenic, tissue repairing monocytes (CD163). IL-6 signals were suppressed in MFP treated mice. (B) MFP-mediated anti-inflammatory activity was via NFκB pathway. (C) MFP treated macrophage showed an increase MMP-9 expression.

FIG. 8 shows the cell death effect of H9c2 cells, (A) PI/Annexin V analysis by flow cytometry, and (B) Caspase-3 expression evaluation by western blot.

FIG. 9 shows a schematic diagram of nanoparticles formation.

FIG. 10 shows TEM micrographs of the formed nanoparticles.

FIG. 11 shows fluorescence microscopes images showing P-selectin targeting effect in HUVEC.

FIG. 12 shows the amino acid sequences of the angiopeptide.

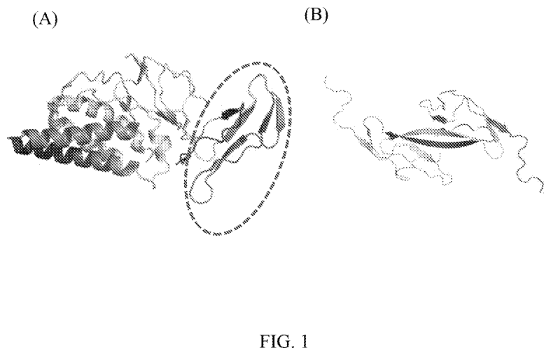

### DETAILED DESCRIPTION OF THE EMBODIMENTS

The present application describes methods and composition for treatment of ischemia disease.

As used herein, the term “ischemic disease” or “ischemia” refers to diseases and/or conditions caused by the reduction in blood flow into a tissue or an organ. Ischemic disease or ischemia includes, for example but not limited to, myocardial infarction, ischemic heart disease, critical limb ischemia, coronary artery disease, cardiac ischemia, angina, congestive heart failure, reperfusion injury, stroke, peripheral artery disease, peripheral vascular disease, transient ischemic attack, brain ischemia, bowel ischemia, intestinal ischemia, mesenteric ischemia, leg ischemia, renal artery disease, diabetic ulcer healing, hepatic ischemia, wound healing, anemia, and atherosclerosis. Some diseases result from ischemia includes, such as, myocardial ischemia, ischemic cardiomyopathy, limb ischemia, cerebrovascular ischemia, pulmonary ischemia and renal ischemia.

An aspect of the present application provides a composition for treatment of ischemia disease comprising: an angiopeptide comprising an amino acid sequence shown as any of SEQ ID Nos: 1 to 3, and a nanoparticle for carrying the angiopeptide.

As used herein, the term “nanoparticle” refers to ultrafine particles which have a diameter between 1 and 1000 nanometres(nm). In embodiments, the nanoparticle of the composition has a mean particle size of 1, 5 10, 50, 100, 200, 300, 400, 500, 600, 700, 800, 900, 950, 1000 nm, or a value between any two of the above points. In one embodiment, the nanoparticle has a mean particle size of between about 50 nm and about 750 nm. In embodiments, the nanoparticle can have a mean particle size of about 50-500 nm, about 100-500 nm, about 150-400 nm, about 150-350 nm, or about 100-350 nm.

In embodiments, the nanoparticle targets inflammatory tissues and/or ischemic tissues. In one embodiment, the nanoparticle targets inflammatory tissues and/or ischemic tissues overexpressing p-selectin.

In embodiments, the nanoparticle is a self-assembled complex nanoparticle. In one embodiment, the self-assembled complex nanoparticle is formed by fucoidan and thermolysin-hydrolyzed protamine peptide.

Another aspect of the present application provides a composition for treatment of ischemia disease comprising: an angiopeptide comprising an amino acid sequence shown as any of SEQ ID Nos: 1 to 3; and a carrier for carrying the angiopeptide.

The carrier can be a hydrogel. In embodiments, the carrier comprises a hybrid multicomponent hydrogel (sometimes referred as “hydrogel system” hereinafter) composed of chemically, morphologically, and functionally different building blocks. The building block can include hyaluronic acid, glucan, cellulose, alginic acid, fucoidan, chitosan, starch, gelatin, collagen, polypeptide, polyacrylic acid, polymethacrylic acid, polyacrylamide, their salts or derivatives, and the like.

In one embodiment, the hydrogel comprises fucoidan, hyaluronic acid, gelatin or any combination thereof. In one embodiment, the hydrogel is a fucoidan-hyaluronic acid, gelatin (FD-HA-GLT) hybrid hydrogel system.

In the present application, the angiopeptide is a haptoglobin subunit, which can be selected from a haptoglobin al subunit, a haptoglobin α2 subunit, or a haptoglobin β subunit. Preferably, the angiopeptide comprises an amino acid sequence of any of SEQ ID Nos: 1 to 3, or an amino acid sequence that is functionally equivalent to any of SEQ ID Nos: 1 to 3.

Functionally equivalent angiopeptide include those showing at least 80%, at least 90%, at least 95%, at least 96%, at least 97%, at least 98% or at least 99% sequence identity with respect to the angiopeptide sequences indicated above, i.e. amino acid sequence of any of SEQ ID Nos: 1 to 3. The degree of identity between two amino acid sequences can be determined by conventional methods, for example, BLAST. The person skilled in the art will understand that the amino acid sequences referred to herein can be modified, for example, by means of chemical modifications that are physiologically relevant, such as, phosphorylations, acetylations, etc.

Another aspect of the present application provides a method for treating and/or preventing ischemia disease in a subject comprising administering a composition to the subject, wherein the composition comprises an angiopeptide comprising an amino acid sequence shown as any of SEQ ID Nos: 1 to 3.

In some embodiments, the composition further comprises a carrier for carrying the angiopeptide. The carrier can be a nanoparticle, a hygrogel or a combination thereof. In one embodiment, the angiopeptide is carried by a nanoparticle, wherein the nanoparticle targets inflammatory tissues and/or ischemic tissues overexpressing p-selectin. In one preferred embodiment, the nanoparticle is formed by fucoidan and thermolysin-hydrolyzed protamine peptide. In one preferred embodiment, the angiopeptid is carried by a hydrogel, wherein the hydrogel comprises fucoidan, hyaluronic acid, gelatin or any combination thereof.

The composition may be administered to the subject via, for example but not limited to, injection, transplantation, transdermal delivery, sublingual administration and the like.

### EXAMPLES

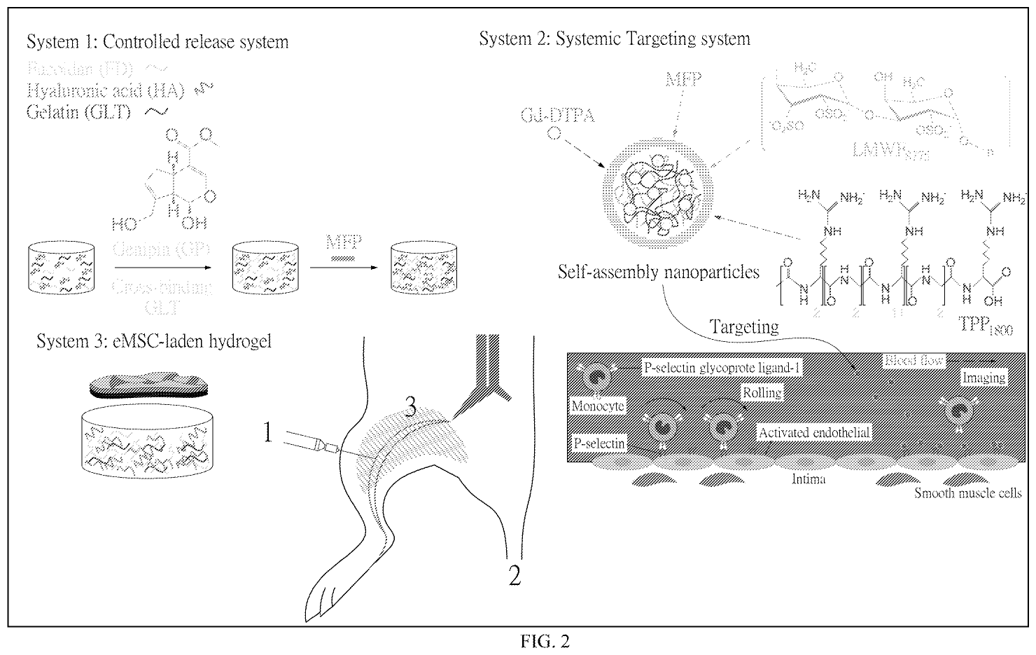

FIG. 2 shows the development of ischemia treatment of the present application including the control release system (hydrogel), the systemic targeting system (nanoparticle), and the eMSC laden hydrogel system. The detail of the embodiments of the three system is described as follows.

### Example 1

Angiopeptide-α1 Purification

The alpha 1-, alpha 2-, and beta- and alpha-beta subunits will be cloned and expressed by using an Escherichia coli (E. coli) expression system. The protein will be following purified by immobilized metal affinity chromatography.

MI Model Preparation and Angiopeptide-α1 Administration

MI will be performed in male C57BL/6 mice (aged 8 weeks; weight, 20-25 g), according to the previous report. Briefly, animals will be anesthetized and underwent tracheal intubation. Operations will be performed by left thoracotomy and ligating the left anterior descending coronary artery with a 6-0 nondestructive suture. Successful MI model will be confirmed by hemodynamic parameters recorded by electrocardiography. The chest will be carefully closed with a 3-0 sterile suture after the lungs will be fully inflated, and incisions will be cleaned and disinfected. After ligation the mice will be immediately randomized to receive of 20 μl angiopeptide-α1 at dose 100 μg/ml, 200 ug/ml, 500 ug/ml and 1000 ug/ml or saline by three injections into three areas adjacent to the infarcted tissue with a 30-gauge needle, then the chest will be immediately closed and spontaneous breathing will be restored.

Myocardial Infarct Size Measurements

Myocardial infarct size will be detected by triphenyl tetrazolium chloride (TTC) and Evans blue dye staining. After 24 h of reperfusion, 2% Evans Blue dye will be injected into the aorta. Then, the hearts will be quickly frozen at 80° C. and will be cut into 2-mm thick slices. The slices will be stained with 1% TTC at room temperature for 30 min. The area will be stained blue by Evans blue indicated the area not at risk, whereas the unstained tissues will be represented AAR. AAR but viable tissue will be stained red by TTC, while the infracted myocardium will be not stained by any dye and will be appeared whiter than other areas. IS will be calculated by image pro plus software as the ratio of IS vs. AAR.

Echocardiography

An ultrasound machine will be adopted to determine the ejection fraction (EF), left ventricular end-diastolic pressure (LVEDP), and left ventricular systolic pressure (LVSP).

Histological Assessments and Immunohistochemical Analysis

The heart will be rinsed with PBS buffer and then perfused with 10% phosphate-buffered formalin. At the end of the perfusion, all hearts will be collected and fixed in 4% paraformaldehyde (PFA) solution for more than 48 h, then paraffin embedded and cut into five micrometer sections. Cross-sections will be analyzed by hematoxylin/eosin (H&E) for morphology; masson's trichrome for collagen deposition and CD31 for neovascularization.

Myocardial Injury Markers

Serum sample will be isolated and stored at 80° C. for further analysis. Serum levels of creatine kinase-MB (CK-MB), lactate dehydrogenase (LDH), and aspartate aminotransferase (AST) will be determined using commercial enzyme-linked immunosorbent (ELISA) assay kits according to the manufacturer's instructions.

Terminal Deoxynucleotidyl Transferase dUTP Nick End Labeling (TUNEL)

TUNEL staining and DAPI will be used to detect nuclear fragmentation following the manufacturer's guidelines (Roche). A total of 2000-3000 cells in 2-3 chosen randomly fields in the border areas of the infarct myocardia from each microslide will be counted to semi-quantitatively identify the ratio of the apoptotic nuclei.

Cell Culture

Stable cell lines order from ATCC, involved monocytes, smooth muscle cells, and HUVECs, and H9c2 cells will be checked the oxidative stress and inflammation relative proteins expression by immunocytochemistry staining and Q-PCR. Human cell lines will be cultured in DMEM with 10% (v/v) fetal bovine serum (FBS) (Gibco, Carlsbad, CA). Human acute monocytic leukemia cells (THP-1) will be cultured in RPMI1640 medium, Gibco, Carlsbad, CA), supplemented with 10% (v/v) FBS. Human umbilical vein endothelial cell (HUVECs) will be cultured in M199 medium (Gibco, Carlsbad, CA), supplemented with 20% (v/v) FBS, and endothelial cell growth supplement (25 μg/mL) (Upstate Biotechnology, Lake Placid, NY). H9C2 will be cultured in DMEM-F12, supplemented with 10% (v/v) FBS. All cultured cell will be maintain in a 10-cm culture dish in a humidified atmosphere of 95% air and 5% CO2 at 37° C. All the cultures above contain penicillin (100 U/mL) and streptomycin (100 μg/mL).

Cell Viability Assay

Cells will be washed with sterile PBS, and 1 ml of DMEM/Hi culture media containing 5 μM CM-H2DCFDA will be added. Cells will be incubated at 37° C. for an additional 30 min and then will be washed again; then, 1 ml of PBS will be added per well. ROS concentrations were determined by FCM and fluorescence microscopy. For FCM detection, each group will be tested in triplicate.

In Vitro Angiogenesis Assay

In vitro tube formation assay will be performed using according to the manufacturer's instructions. Human umbilical vein endothelial cells (HUVECs) will be seeded at 1×10⁴ per well of 96-well plates and will be treated with either Hp peptide. Tube formation will be examined by phase-contrast microscopy after 6 h. Quantification will be done using image analysis software (ImageJ). We will calculate the numbers of tubes per field, branch points per field, and the tube area per field in at least five images from different areas of each sample in three independent experiments.

Annexin V-PE/7AAD Assay

Annexin V-PE/7-ADD will be detected by FCM to measure apoptosis in H9c2. A total of 5×10⁵ H9c2s will be seeded in 6-well plates overnight. Briefly, cells will be collected, washed twice with ice-cold PBS, and incubated for 15 min in 1× Annexin V Binding Buffer containing 7-AAD-Percp and Annexin V-PE. Finally, apoptosis will be detected by FCM.

Autophagic Flux Measurements

To detect autophagic flux, the mRFP-GFP-LC3 reporter plasmid (1 μL/mL) will be transfected into H9c2s using lipofectamine 2000 according to the manufacturer's instructions. Then, the transfected cells will be processed and grouped as described above. The cell Images will be obtained using a confocal microscopy and autophagosome and autolysosome dots will be quantified manually in at least 4 different H9c2 per group.

Immunofluorescence Microscopy and Confocal Microscopy

The specimens of tissues will be immunohistochemistry stained. Or treated cells will be seeded on glass cover slides for immunofluorescence staining. After 24 h cultured, the cells will be fixed with 4% paraformaldehyde for 1 h at 37° C. incubator. Following wash with phosphate buffered saline (PBS), the cell will be incubated with blocking buffer (10% FBS and 0.25% Triton X-100 dissolved in PBS) in 37□ for 1 h. Wash with PBS (containing 0.25% Triton X-100). The fluorescence-conjugated anti-CD44 will be treated for 1 h at 37□. After washing with PBS 3 times, nucleus will be stained with Hoechst 33342 (Invitrogen, Burlington, Ontario, Canada). Capture cell fluorescence images by Olympus BX51 microscope.

RNA Isolation and Quantitative Real-Time PCR (qRT-PCR)

Total RNA from heart tissues and H9c2 cells will be extracted using Trizol Reagent. Quantitated RNA (1 μg) will be used for generating cDNA by using the Reverse Transcription System Kit. The levels of mRNAs and miRNAs will be determined using SYBR Green on a 7500 Fast Real-Time Sequence detection system. GAPDH will be used as a reference gene. The relative levels of gene expression will be assessed using the 2−ΔΔCt method. All samples will be analyzed in triplicate.

Western Blot Assay

Total protein from heart tissues and H9c2 cells will be extracted using RIPA lysis buffer. Equal amounts of protein will be separated by 10%-12% SDS-PAGE and then transferred electrophoretically to PVDF membranes. After blocked with 5% nonfat milk for 1 h at room temperature, the membranes will be incubated with the specific primary antibodies at 4° C. overnight. After washed three times with TBST, the membranes will be incubated with horseradish peroxidase-conjugated secondary antibodies for 1 h at room temperature. The signals will be visualized using an enhanced chemiluminescent detection kit. The data will be normalized to GAPDH as an internal control.

Statistical Analysis

Quantitative results will be expressed as mean±standard deviation (SD) from at least three separate experiments. Comparisons between groups will be made by Student's t-tests or one-way analysis of variance with Tukey's post hoc tests. P value<0.05 will be considered statistically significant.

Result

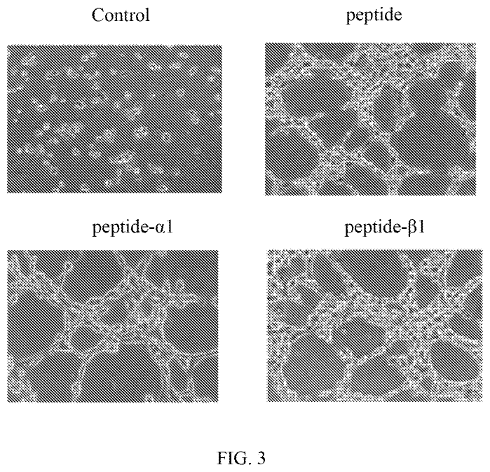

FIG. 3 shows angiogenesis ability of the angiopeptide used in the present application. All the phenotypes of angiopeptide have the ability of capillary formation.

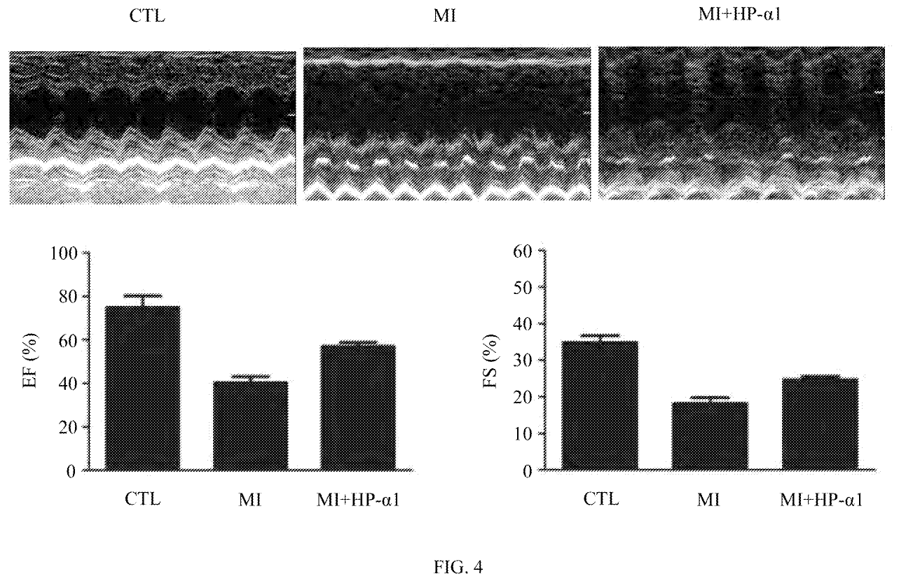

FIG. 4 shows successful preparation of the MI model. EF and FS were measured by echocardiography.

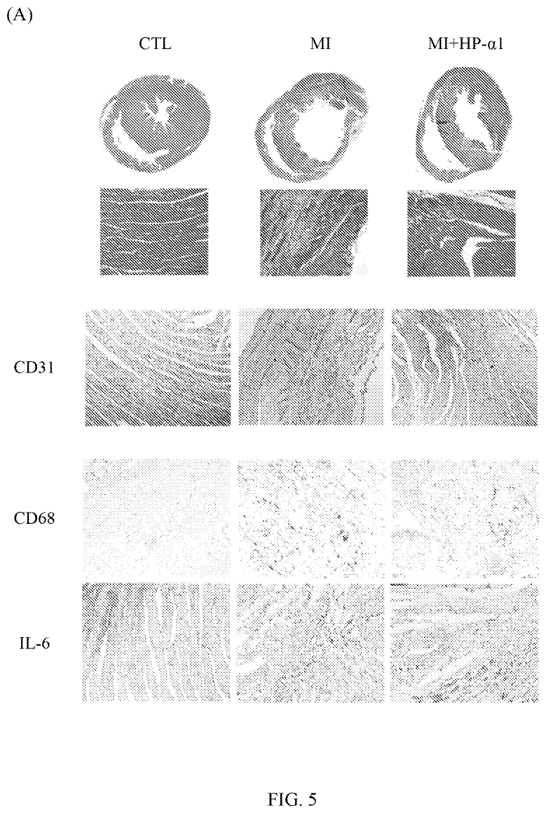

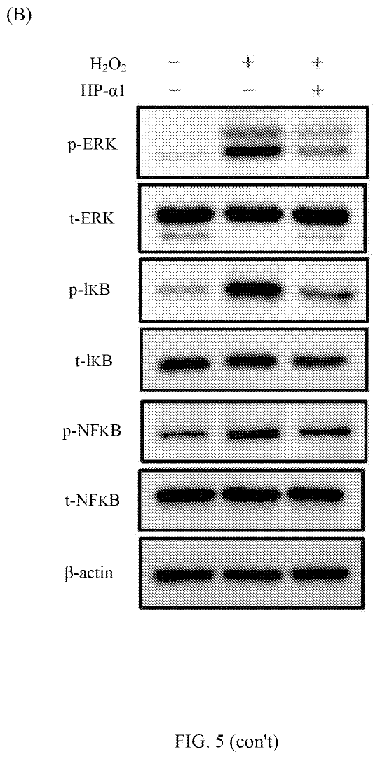

In the test of the left anterior descending artery (LAD) ligated rats, FIG. 5(A) shows representative histological and immunohistochemical staining for Masson trichrome, CD31, and IL-6 for evaluation the effects of the angiopeptide on fibrosis, angiogenesis, and inflammation. The angiopeptide decreased the heart fibrosis and IL-6 expression, whereas enhanced CD31 expression in LAD ligated rat. In FIG. 5(B), H9C2 cells were pre-treated the angiopeptide following H₂O₂ stimulation. Western blot was performed to analyze the expression of p-ERK/ERK, p-IkB/IkB, and p-NF-kB/NF-kB. The results suggest the angiopeptide ameliorated myocardial infarct size, involving in up-regulated angiogenesis and down-regulated inflammation.

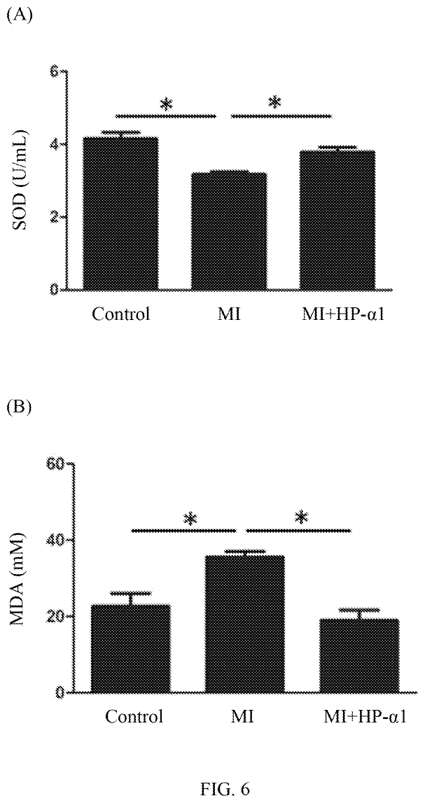

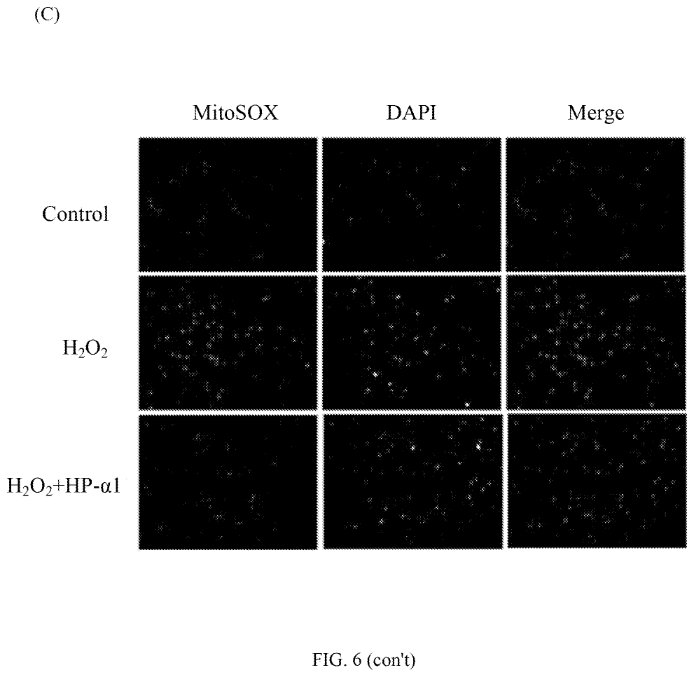

FIG. 6 shows that (A) SOD activity and (B) MDA level were measured using ELISA kit in the heart homogenates from LAD ligated rats. *P<0.05 was considered significant. In FIG. 6(C), H9C2 cells were pre-treated the MFP following H₂O₂ stimulation. Mitochondrial ROS was detected using MitoSOXRed assay. Treatment with the MFP suppressed in H₂O₂-induced mitochondrial ROS production in cardiomyocytes. The results suggest the MFP inhibited oxidative stress in myocardial infarction.

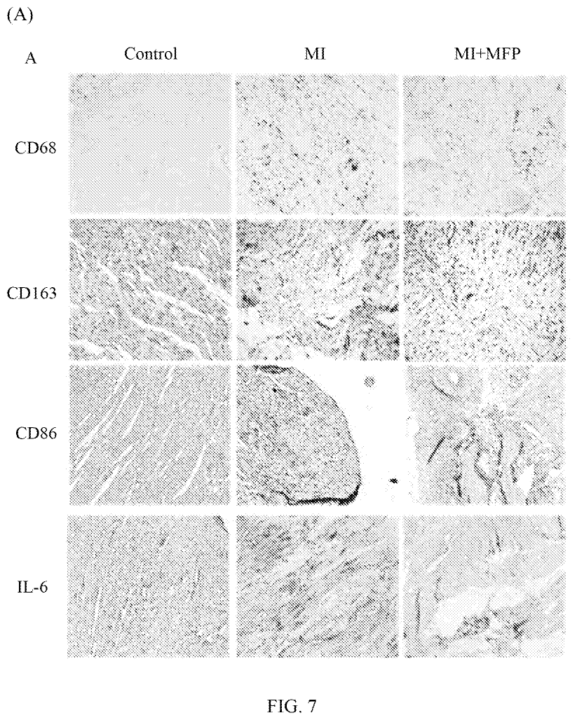

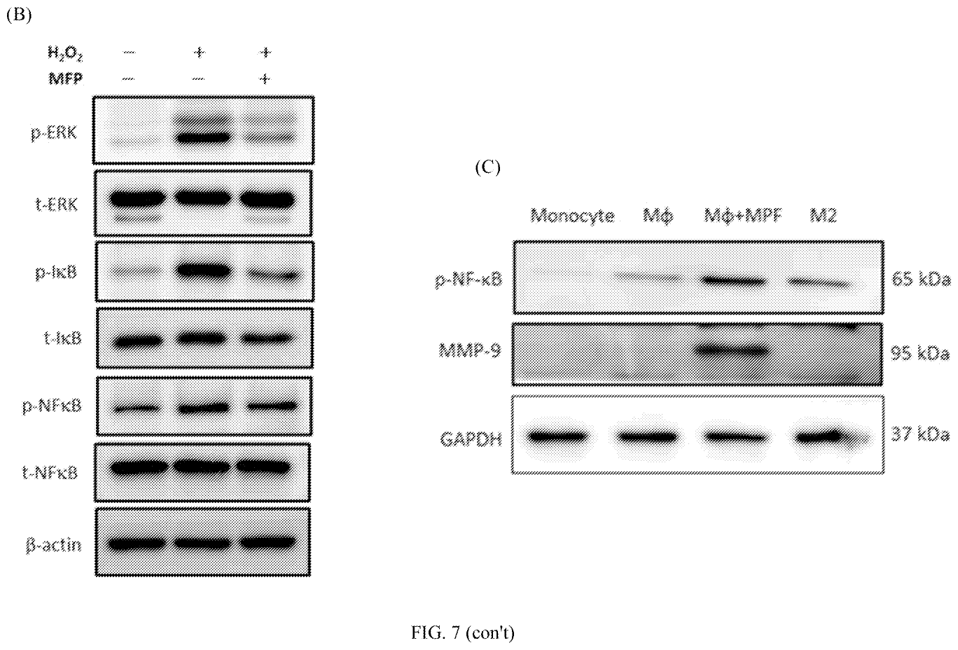

FIG. 7 shows that (A) MFP lowered monocyte (CD68) infiltration and increased infiltration of angiogenic, tissue repairing monocytes (CD163). IL-6 signals were suppressed in MFP treated mice. (B) MFP-mediated anti-inflammatory activity was via NFκB pathway. (C) MFP treated macrophage showed an increase MMP-9 expression.

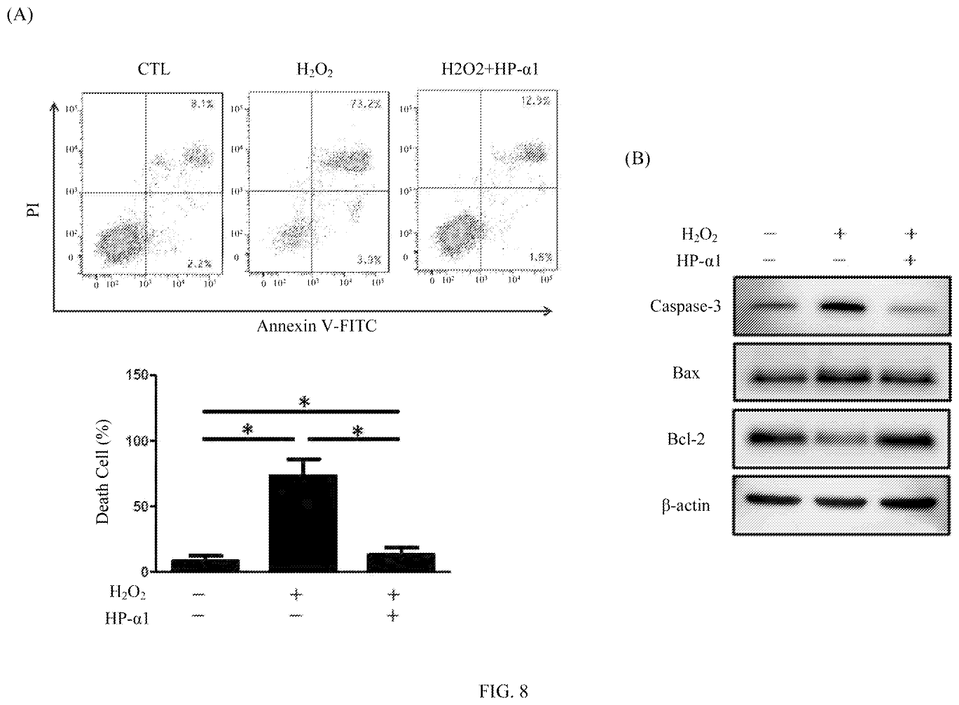

FIG. 8 shows the cell death effect of H9c2 cells, (A) PI/Annexin V analysis by flow cytometry, and (B) Caspase-3 expression evaluation by western blot.

### Example 2

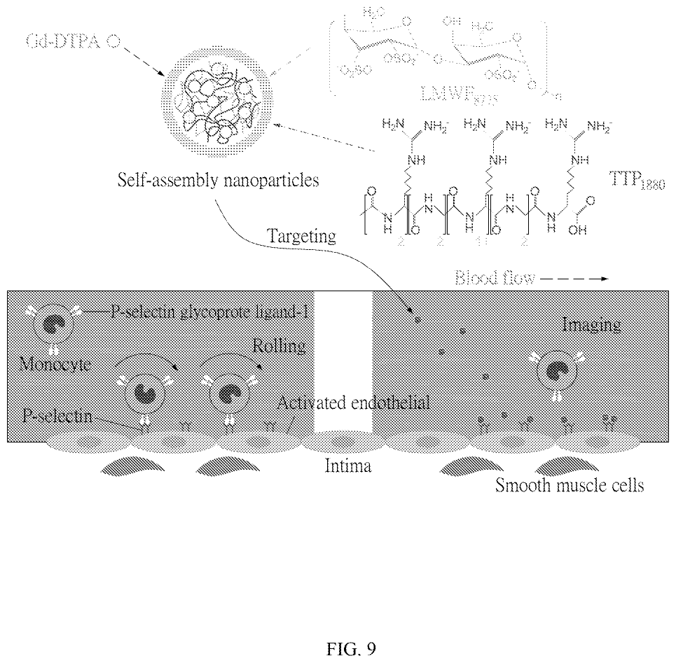

FIG. 9 shows a schematic diagram of nanoparticles formation and P-selectin targeting. The detail process is described as follows.

Preparation of Nanoparticles (NPs)

LMWP-P-selectin will be also prepared with an expression plasmid, which overproduced a protein containing LMWP-P-selectin in Escherichia coli. Briefly, as the first template, complementary cDNAs for LMWP-P-selectin will be constructed by serial polymerase chain reaction (PCR)-mediated addition of the LMWP codons to the p-selectin genes. Subsequently, the p-selectin gene fragments will be amplified by PCR. Then, the resulting PCR products will be subcloned into a TOPO vector. After plasmid purification and digestion with NdeI/XhoI, the sequence will be confirmed by capillary sequencing and then cloned into the pET-41b(+) expression vector. pET-41b(+)-LMWP-p-selectin will be transformed into competent E. coli cells (BL21-DE3) and cultured at 37° C. in potassium-modified Luria broth (LBK) medium supplemented with 100 μg/mL ampicillin until the optical density of the culture at 600 nm will be 0.6-0.8. Protein expression will be induced by adding 1 M isopropyl-β-D-thiogalactopyranoside (IPTG), and the cells were grown overnight at 25° C. with shaking at 100 rpm. The cells will be collected by centrifugation (6,000×g for 10 minutes at 4° C.), resuspended in cell lysis buffer (sodium phosphate buffer, pH 7.5, containing 10 mM NaCl), and sonicated at 50% amplitude following a 10/10-second sonication/holding cycle. The supernatant, containing LMWP-p-selectin, will be collected by centrifugation (12,000×g, 10 minutes, 4° C.) and filtered through a 0.45 μm membrane (EMD Millipore, Billerica, MA, USA) before further analysis. The NPs will be made by Cy5-conjugated for imaging.

Attachment of Angiopeptide-α1 to Nanoparticles (Angiopeptide-α1 NPs)

Angiopeptide-α1 (56 mg) in dimethylsulfoxide (DMSO; 2 mL) will be added to a solution of nanoparticles from poly (valerolactoneepoxyvalerolactone-allylvalerolactone-oxepanedione) containing 11% epoxide and cross-linked with 1 equivalent of 2,2-(ethylenedioxy)bis (ethylamine) per epoxide (ref. 11; 105.6 mg, 0.78 μmol) in DMSO (1 mL). The reaction mixture will be heated for 72 hours at 34° C. Residual peptide will be removed by dialyzing with SnakeSkin Pleated Dialysis Tubing (molecular weight cutoff 10,000) against 50/50 THF/CH3CN.

Mouse MI Model and Angiopeptide-α1 NPs Injection

MI will be performed in male C57BL/6 mice (aged 8 weeks; weight, 20-25 g), according to the previous report. Briefly, animals will be anesthetized and underwent tracheal intubation. Operations will be performed by left thoracotomy and ligating the left anterior descending coronary artery with a 6-0 nondestructive suture. Successful MI model will be confirmed by hemodynamic parameters recorded by electrocardiography. The chest will be carefully closed with a 3-0 sterile suture after the lungs will be fully inflated, and incisions will be cleaned and disinfected. Within 30 mins after MI, the mice will be intravenously injected angiopeptide-α1 NPs via the tail vein and mice will be randomly set into 4 groups: control-NPs, 2.5 mg/kg angiopeptide-α1-NPs, 5 mg/kg angiopeptide-α1-NPs, 20 mg/kg angiopeptide-α1-NPs, and 30 mg/kg angiopeptide-α1-NPs.

Immunofluorescence Microscopy and Confocal Microscopy

To estimate the Cy5-conjugated angiopeptide-α1 NP homing to injury cells in vitro and in vivo, immunofluorescence microscopy and confocal microscopy will be applied. The specimens of tissues or treated cells will be put on glass cover slides for immunofluorescence microscopy. Nucleus will be stained with Hoechst 33342 (Invitrogen, Burlington, Ontario, Canada). Confocal microscopy will be used to evaluate NPs' locolization.

Isolation, Culture, and Characterization of Mouse EPCs

Mouse EPCs will be isolated and cultured as described previously. Briefly, the bone marrow will be extracted from the femora and tibiae of C57BL/6 mice of 4 weeks old. Bone marrow-mononuclear cells will be further layered by density gradient centrifugation (Histopaque 1083, Sigma-Aldrich, USA). After being washed twice, cells will be seeded onto a culture dish and cultured in EGM-2 MV (Lonza, Switzerland) supplemented with 5% FBS. Then, the cells will be incubated at 37° C. in a humidified atmosphere containing 5% CO₂. The culture medium will be refreshed every 48 h. When grown to 90% confluence, cells will be harvested with 0.25% trypsin (Sigma-Aldrich, USA) and passaged continuously. The phenotypes of EPCs will be examined by flow cytometry using antibodies against mouse CD11b, CD31, CD45, CD133, VE-cadherin, and Flk-1, respectively (Sigma, USA). DiI-ac-LDL uptake assay will be conducted to further investigate the characteristics of EPCs.

Myocardial infarct size measurements, Echocardiography, Histological assessments and immunohistochemical analysis, Myocardial injury markers, Terminal deoxynucleotidyl transferase dUTP nick end labeling (TUNEL), Western blot assay and Statistical analysis are conducted as described in Example 1.

Result

The depolymerized low molecular weight fucoidan (LMWF8775 Da) and a thermolysin-hydrolyzed protamine peptide (TPP1880 Da) were able to form self-assembled complex nanoparticles (CNPs). The CNPs selectively targeted PMA-stimulated, inflamed endothelial cells (HUVECs) with high expression of P-selectin. Gd-DTPA MRI contrast agent was successfully loaded in the CNPs with better T1 relaxivity and selectively accumulated in the activated HUVECs with increased MRI intensity and reduced cytotoxicity as compared to free Gd-DTPA.

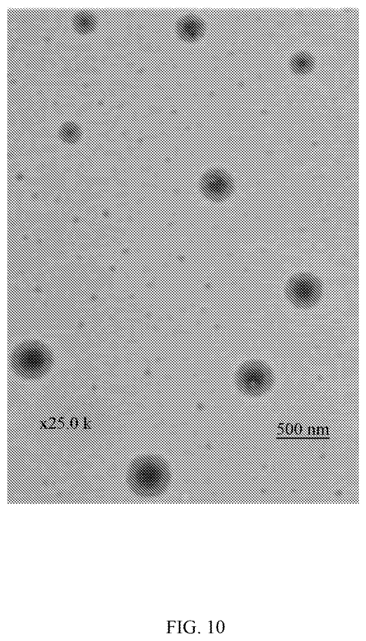

FIG. 10 shows TEM micrographs of the formed nanoparticles, i.e. Gd-DTPA loaded LMWF8775 Da-based CNPs. The scale bar in the figures is 500 nm. The formed nanoparticles have a diameter of about 100 nm to about 350 nm.

The results suggest that the TPP1880 Da/LMWF8775 Da CNPs have potential for early diagnosis of cardiovascular diseases and cancers in which the endothelium is inflamed or activated.

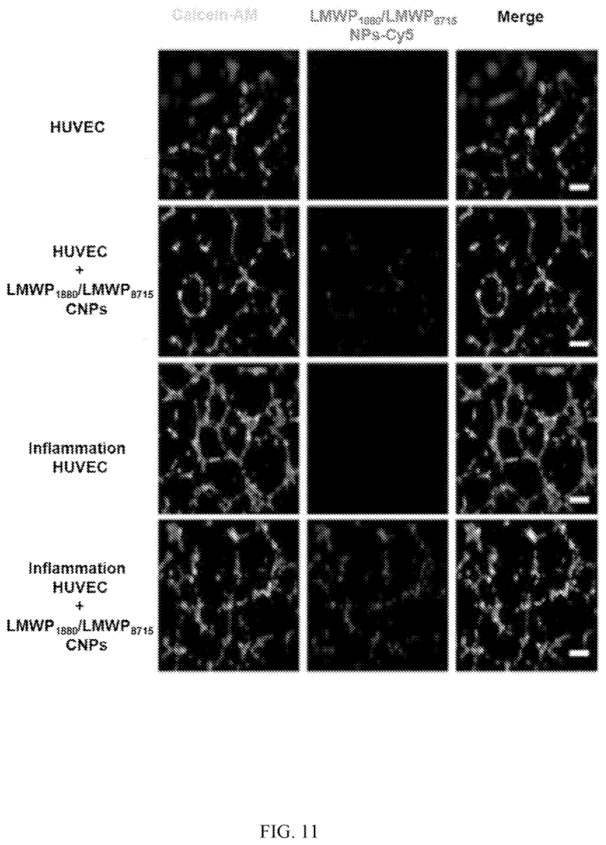

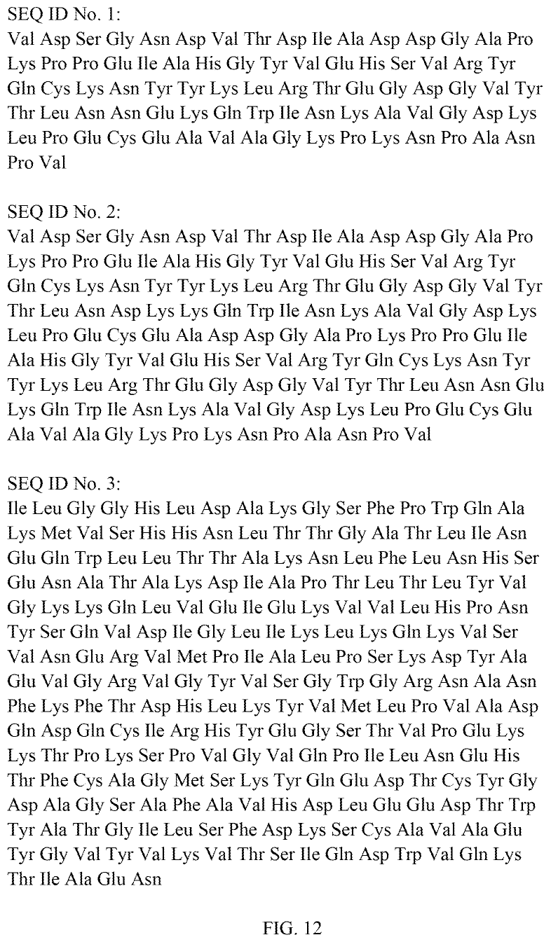

FIG. 11 shows fluorescence microscopes images showing P-selectin targeting effect of LMWP/LMWF NPs in PMA-induced inflammation HUVEC and angiopeptide-α1-induced HUVEC angiogenesis.

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims and its equivalent systems and methods.

What is claimed is:

1. A composition for treatment of ischemia disease comprising:
an angiopeptide comprising an amino acid sequence shown as any of SEQ ID Nos: 1 to 3; and
a nanoparticle for carrying the angiopeptide.

2. The composition of claim 1, wherein the nanoparticle is a self-assembled complex nanoparticle.

3. The composition of claim 1, wherein the nanoparticle targets inflammatory tissues and/or ischemic tissues overexpressing p-selectin.

4. The composition of claim 2, wherein the self-assembled complex nanoparticle is formed by fucoidan and thermolysin-hydrolyzed protamine peptide.

5. The composition of claim 1, wherein the nanoparticle has a mean particle size of between about 50 nm and about 750 nm.

6. A composition for treatment of ischemia disease comprising:
an angiopeptide comprising an amino acid sequence shown as any of SEQ ID Nos: 1 to 3; and
a carrier for carrying the angiopeptide.

7. The composition of claim 6, wherein the carrier is a hydrogel.

8. The composition of claim 7, wherein the hydrogel comprises fucoidan, hyaluronic acid, gelatin or any combination thereof.

9. The composition of claim 7, wherein the hydrogel is a hybrid hydrogel of fucoidan, hyaluronic acid and gelatin.

10. The composition of claim 6, further comprising a nanoparticle.

11. A method for treating and/or preventing ischemia disease in a subject comprising administering a composition to the subject, wherein the composition comprises an angiopeptide comprising an amino acid sequence shown as any of SEQ ID Nos: 1 to 3.

12. The method of claim 11, wherein the composition further comprises a nanoparticle for carrying the angiopeptide, wherein the nanoparticle targets inflammatory tissues and/or ischemic tissues overexpressing p-selectin.

13. The method of claim 12, wherein the nanoparticle is formed by fucoidan and thermolysin-hydrolyzed protamine peptide.

14. The method of claim 11, wherein the composition further comprises a hydrogel carrier for carrying the angiopeptide.

15. The method of claim 14, wherein the hydrogel comprises fucoidan, hyaluronic acid, gelatin or any combination thereof.

16. The method of claim 11, wherein the composition is administered to the subject with via injection, transplantation, transdermal delivery, and/or sublingual administration.

17. The method of claim 11, wherein the ischemia disease comprises myocardial infarction, ischemic heart disease, critical limb ischemia, coronary artery disease, cardiac ischemia, angina, congestive heart failure, reperfusion injury, stroke, peripheral artery disease, peripheral vascular disease, transient ischemic attack, brain ischemia, bowel ischemia, intestinal ischemia, mesenteric ischemia, leg ischemia, renal artery disease, diabetic ulcer healing, hepatic ischemia, wound healing, anemia, and atherosclerosis. Some diseases result from ischemia includes, such as, myocardial ischemia, ischemic cardiomyopathy, limb ischemia, cerebrovascular ischemia, pulmonary ischemia and/or renal ischemia.


SEQUENCE LISTING (3 sequences)

SEQ ID NO: 1 - protein, 82 aa, Artificial Sequence
  angiopeptide
VDSGNDVTDIADDGAPKPPEIAHGYVEHSVRYQCKNYYKLRTEGDGVYTLNNEKQWINKAVGDKLPECEAVAGKPKNPANPV

SEQ ID NO: 2 - protein, 141 aa, Artificial Sequence
  angiopeptide
VDSGNDVTDIADDGAPKPPEIAHGYVEHSVRYQCKNYYKLRTEGDGVYTLNDKKQWINKAVGDKLPECEADDGAPKPPEIAHGYVEHSVRYQCKNYYKLRTEGDGVYTLNNEKQWINKAVGDKLPECEAVAGKPKNPANPV

SEQ ID NO: 3 - protein, 245 aa, Artificial Sequence
  angiopeptide
ILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPIALPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN